(19) United States
(12) Patent Application Publication
(10) Pub. No.: US 2020/0297225 A1
(43) Pub. Date: Sep. 24, 2020

(54) VITAL SIGN MEASUREMENT DEVICE

(71) Applicant: AMI INC., Minamata-shi, Kumamoto (JP)

(72) Inventor: Shinpei OGAWA, Minamata-shi, Kumamoto (JP)

(21) Appl. No.: 16/616,492

(22) PCT Filed: Aug. 29, 2018
(86) PCT No.: PCT/JP2018/031884

§ 371 Date: Nov. 24, 2019

(30) Foreign Application Priority Data
Aug. 29, 2017 (JP) 2017-163947

(51) Int. Cl. A61B 5/021; A61B 5/01; A61B 5/00; A61B 5/08; A61B 5/022; A61B 5/0225

(52) U.S. Cl. CPC A61B 5/02141; A61B 5/01; A61B 5/02255; A61B 5/08; A61B 5/02225; A61B 5/6823

(57) ABSTRACT

To easily and accurately acquire an electrocardiogram waveform in a device for simultaneously measuring blood pressure, an electrocardiogram, and other vital signs. A vital sign measurement device 100 is provided with a blood pressure measurement cuff 20 for pressing on a certain measurement part of a subject, one or a plurality of biosignal sensors 30, 40 for detecting a biosignal of a separate measurement part of the subject, a plurality of electrodes 51-54 for contacting the skin of the subject and detecting physical electrical potentials, and a device body 10. The device body 10 measures blood pressure of the subject by increasing and decreasing the cuff pressure in the cuff 20, measures vital signs other than the blood pressure and electrocardiogram of the subject on the basis of biosignals detected by the biosignal sensors 30, 40, and measures the electrocardiogram of the subject on the basis of the physical electrical potentials detected by the plurality of electrodes 51-54. At least one of the plurality of electrodes is provided to the cuff 20, and at least one of the plurality of electrodes is provided to the biosignal sensors 30, 40.

### TECHNICAL FIELD

The present invention relates to a vital sign measurement device for simultaneously measuring vital signs including a blood pressure and an electrocardiogram of a subject.

### BACKGROUND ART

Conventionally, in medical setting, a device that measures various vital signs of a subject such as a blood pressure, an electrocardiogram, a heart rate, a body temperature, and a blood oxygen saturation level has been used. These vital signs are very important in the medical setting, but generally, these vital signs are separately measured by type.

On the other hand, Patent Document 1 discloses a remote diagnostic device configured to simultaneously measure vital signs including the blood pressure and the electrocardiogram. This device is configured such that an electrocardiogram measurement electrode and a blood pressure and heart rate measurement device are mounted on a glove member adaptable to be worn on a person's hand, and such biosignals can be detected by applying this glove member to a chest of a subject.
Patent Document 1: Pamphlet of WO/1999/060919

### DISCLOSURE OF THE INVENTION

### Problems to be Solved by the Invention

However, in the glove-type diagnostic device disclosed in Patent Document 1, for the electrocardiogram measurement, a distance between the electrodes can be separated only by a size of a palm of the hand at maximum, thus having a problem that an electrocardiogram waveform cannot be accurately measured since an electric potential difference between the electrodes is small. The electrocardiogram measurement electrode has to contact a skin of the subject. However, in the device in Patent Document 1 designed on the premise of applying the glove member to the chest, it is necessary to expose the chest of the subject in use. Thus, there is a concern that a usable environment is restricted.

Therefore, an object of the present invention is to easily and accurately acquire an electrocardiogram waveform in a device that simultaneously measures vital signs such as a blood pressure and an electrocardiogram.

### Solutions to the Problems

The inventor of the present invention earnestly examined a solution for the above-described problem of the conventional invention, and as a result, acquired an knowledge that a blood pressure measurement cuff and a biosignal sensor for measuring other vital signs are provided, at least one of electrocardiogram measurement electrodes is mounted on the cuff, and other electrodes are mounted on the biosignal sensor, thus ensuring simultaneous measurement of at least three kinds of vital signs including the blood pressure and the electrocardiogram and easily keeping a distance between the electrodes to ensure accurate measurement of an electrocardiogram waveform. Then, the inventor thought that the problem of the prior art can be solved based on the above-described knowledge and completed the present invention. Specifically, the present invention has the following configuration.

The present invention relates to a vital sign measurement device. The vital sign measurement device according to the present invention includes a blood pressure measurement cuff, one or a plurality of biosignal sensors, a plurality of electrocardiogram measurement electrodes, and a device body coupled to them. The device body measures a blood pressure of a subject by increasing and decreasing a cuff pressure in the cuff. The device body measures a vital sign other than the blood pressure or an electrocardiogram of the subject based on a biosignal detected by the biosignal sensor. “The vital sign other than the blood pressure or the electrocardiogram” includes various vital signs such as a pulse, a blood oxygen saturation level, a heartbeat, a body temperature, a heart sound, a brain wave, a respiratory sound, and a respiration rate. Thus, as the biosignal sensor, a known one for measuring these vital signs can be appropriately employed. The device body further measures the electrocardiogram of the subject based on physical electrical potentials detected by the plurality of electrodes. At least one of the plurality of electrodes is provided to the cuff (a part contacting the skin of the subject), and at least another of the plurality of electrodes is provided to the biosignal sensor (a part contacting the skin of the subject).

As the above-described configuration, providing the respective electrocardiogram measurement electrodes to the blood pressure measurement cuff and the biosignal sensor for measuring other vital signs facilitates sufficiently separated distance between the electrodes, thus ensuring accurate measurement of the electrocardiogram. The cuff is generally wound around one arm of the subject. For example, while the cuff is mounted on one arm, the biosignal sensor can be operated by another arm, thus facilitating use of the measurement device by the subject himself/herself. What is called, a I Induction can be detected by detecting the physical electrical potentials of the arm on which the cuff is mounted and the arm gripping the biosignal sensor with the electrodes, thus having a sufficient electric potential difference to be effective for detection of an arrhythmia. Further, simultaneously with the electrocardiogram, the blood pressure and the other vital signs can be measured.

In the present invention, the biosignal sensor preferably includes a pulse oximeter probe. The probe irradiates a biological tissue having a bloodstream of the subject with a light to detect optical information of a transmitted light or a reflected light. In this case, the device body measures at least any one of a blood oxygen saturation level and a pulse of the subject based on the optical information detected by the probe. With such a configuration, the blood oxygen saturation level and the pulse can be simultaneously measured in addition to the blood pressure and the electrocardiogram.

In the present invention, the biosignal sensor may further include a thermometer. With it, the body temperature of the subject can be simultaneously measured in addition to the blood pressure, the electrocardiogram, the blood oxygen saturation level, and the pulse.

In the present invention, the biosignal sensor preferably further includes a digital stethoscope chest piece. The chest piece includes a microphone that converts a heart sound of the subject into an electrical signal. With such a configuration, the heart sound and the respiratory sound of the subject can be simultaneously measured in addition to the blood pressure, the electrocardiogram, the blood oxygen saturation level, and the pulse.

In the present invention, it is preferable that any one of the plurality of electrodes is provided to a part contacting the skin of the subject in the probe, and another of the plurality of electrodes is provided to a part contacting the skin of the subject in the chest piece. With such a configuration, a first electrode provided to the cuff, a second electrode provided to the probe, and a third electrode provided to the chest piece ensure measurement of the physical electrical potentials of the subject at three portions, thus improving an accuracy of the electrocardiogram. For example, in addition to a bipolar lead between the first electrode and the second electrode, bipolar leads between the first electrode and the third electrode and between the second electrode and the third electrode can be measured.

In the present invention, the probe and the chest piece to which the respective electrodes are provided may be removably combined. For example, by mounting the chest piece on the probe having a type used by inserting a hand finger into it, the subject can acquire the heart sound and the like by pressing this finger to the chest while fitting the finger to the probe, thus facilitating operation of each piece of equipment. On the other hand, for example, when the auscultation of the heart sound and the like is not necessary, the chest piece can be removed. Alternatively, when the subject cannot lift the chest piece to his/her chest because of a physical reason such as paralysis and contracture, while the pulse oximeter is mounted on the hand finger of the subject, a helper can press the chest piece to the chest of the subject. Thus, the probe and the chest piece can be used by attaching and removing them corresponding to the usage situation.

In the present invention, the device body preferably extracts the pulse wave of the subject from the electrocardiogram and, in a process of decreasing the cuff pressure in the cuff after increasing it, measures a maximum blood pressure and a minimum blood pressure of the subject at a timing corresponding to this pulse wave. This can improve an accuracy of the blood pressure measurement using an electrocardiogram waveform. That is, an automated sphygmomanometer generally measures the maximum blood pressure and the minimum blood pressure of the subject by employing an oscillometric method. However, when the subject is suffering from the low blood pressure or the arrhythmia, the pulse wave is sometimes hard to be sensed to make the blood pressure immeasurable. In this respect, an accuracy of the automated sphygmomanometer can be enhanced by accurately acquiring the timing of the pulse wave of the subject from the electrocardiogram and acquiring the maximum blood pressure and the minimum blood pressure at the timing corresponding to pulsation.

In the present invention, the device body may extract a time slot of both or any one of a systole and a diastole of a heart of the subject from the electrocardiogram and determine whether there is a heart murmur in the heart sound in the time slot (the systole and/or the diastole) extracted from a heart sound signal acquired by the microphone. Such a configuration can automatically acquire the heart murmur of roughly the systole and the diastole of the heart generated in a disease such as an aortic valve stenosis and the like or an aortic valve insufficiency. Determination of the systole or the diastole with reference to electrocardiogram data ensures an accurate and automatic determination of the heart murmur.

In the present invention, the device body discriminates the time slots of the systole and the diastole of the heart of the subject from the electrocardiogram and acquires a difference (that is, a “pulse pressure”) in the blood pressures of the subject in the systole and the diastole. The “pulse pressure” means a difference between a systolic blood pressure and a diastolic blood pressure. When the heart murmur is recognized in the systole, the subject is strongly suspected of suffering from the aortic valve stenosis. This disease has a trend that the heart murmur in the systole increases as a symptom gets worse, but the heart murmur in the systole rather becomes weak as the symptom further becomes severe. This is because, in the severe aortic valve stenosis, a wall of a left ventricle of the heart becomes thick and contraction becomes weak, thus reducing an outflow of blood. Thus, even the heart murmur in the systole is measured, the end-stage aortic valve stenosis may be overlooked. Accordingly, as the above-described configuration, simultaneously measuring the pulse pressure in addition to the heart murmur in the systole can examine the severe aortic valve stenosis from the aspect of the heart murmur and the pulse pressure, thus enhancing an accuracy in diagnosis of disease. Specifically, even when the heart murmur in the systole is lower than a constant threshold, insofar as the pulse pressure is equal to or less than a threshold, it may automatically diagnose that the subject is suspected of suffering from the aortic valve stenosis. Further, when the heart murmur is recognized in the diastole, the subject is strongly suspected of suffering from the aortic valve insufficiency. However, this aortic valve insufficiency, in a severe case, also has a trend that the heart murmur in the diastole becomes weak. Accordingly, also in this case, simultaneously measuring the pulse pressure in addition to the heart murmur in the diastole can examine the severe aortic valve insufficiency from the aspect of the heart murmur and the pulse pressure. Specifically, even when the heart murmur in the systole is lower than a constant threshold, in a case where the pulse pressure exceeds a threshold, it may automatically diagnose that the subject is suspected of suffering from the aortic valve insufficiency.

### Advantageous Effects of the Invention

With the present invention, in the device for simultaneously measuring the vital signs such as the blood pressure and the electrocardiogram, the electrocardiogram waveform can be easily and accurately acquired.

### BRIEF DESCRIPTION OF THE DRAWINGS

FIG. 1 is a schematic diagram illustrating a use state of a vital sign measurement device according to a first embodiment of the present invention.

FIG. 2 is a block diagram illustrating an exemplary configuration of the vital sign measurement device.

FIG. 3 schematically illustrates an exemplary automatic detection of heart murmur.

FIG. 4 illustrates an exemplary automatic diagnosis flow of an aortic valve stenosis.

FIG. 5 illustrates an exemplary automatic diagnosis flow of an aortic valve insufficiency.

### DESCRIPTION OF PREFERRED EMBODIMENTS

The following describes an embodiment of the present invention using the drawings. The present invention is not limited to the embodiment described below and includes ones appropriately changed in an obvious range by those skilled in the art from the following embodiment.

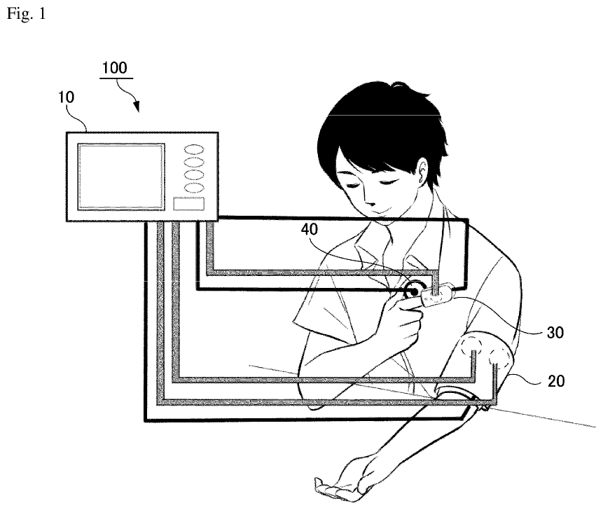

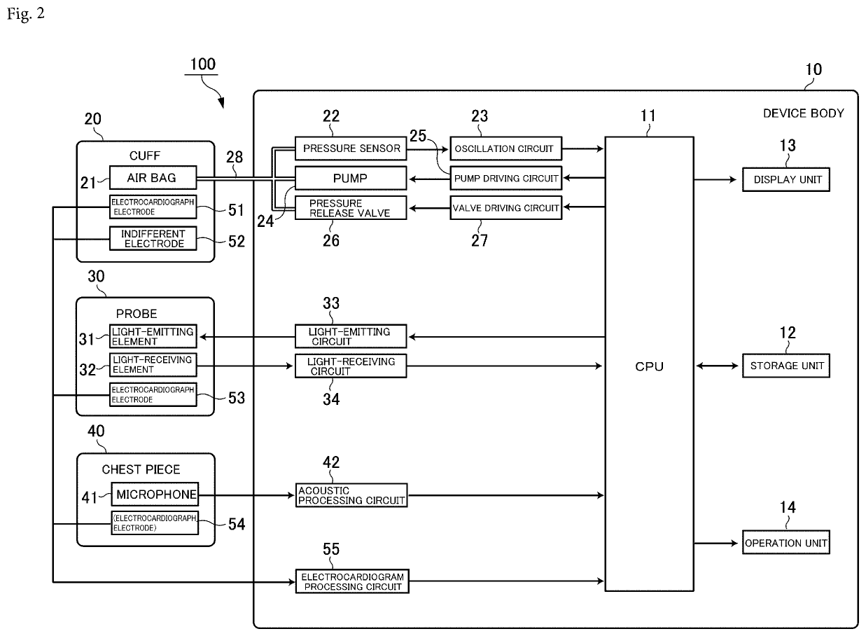

FIG. 1 schematically illustrates a use state of a vital sign measurement device 100 according to a first embodiment. FIG. 2 illustrates an exemplary configuration of the vital sign measurement device 100 illustrated in FIG. 1. As illustrated in FIG. 1 and FIG. 2, the vital sign measurement device 100 includes a device body 10, a blood pressure measurement cuff 20, a pulse oximeter probe 30, and a digital stethoscope chest piece 40. Further, one or a plurality of electrocardiogram measurement electrodes 51, 52, 53, and 54 are mounted on the cuff 20, the probe 30, and the chest piece 40. Thus, the vital sign measurement device 100 according to the embodiment is configured to simultaneously measure vital signs such as a blood pressure, a blood oxygen saturation level, a pulse, a heart sound, a respiratory sound, and an electrocardiogram waveform of a subject.

As illustrated in FIG. 1, the device body 10 includes a central processing unit (CPU) 11, a storage unit 12, a display unit 13, and an operation unit 14 as underlying function blocks. The CPU 11 controls the whole vital sign measurement device 100 by reading a program stored in the storage unit 12 and controlling other components and executing predetermined computing in accordance with this program. The storage unit 12 has a storage function achieved by a non-volatile memory such as an HDD and an SDD. The storage unit 12 may have a function as a memory for writing or reading, for example, an interim progress of computation processing by the CPU 11. The memory function of the storage unit 12 is achieved by a volatile memory such as a RAM and a DRAM. The display unit 13 is a display device such as a liquid crystal display and an organic EL display. The operation unit 14, which is configured from input devices such as a computer mouse, a keyboard, a touch panel, and a microphone, accepts operation information by humans. The display unit 14 may configure a touch panel display integrally with the operation unit 15.

In the measurement device 100 of the present invention, a sphygmomanometer is configured from the cuff 20 having an air bag 21, and the CPU 11, a pressure sensor 22, an oscillation circuit 23, a pump 24, a pump driving circuit 25, a pressure release valve 26, a valve driving circuit 27, and an air hose 28 included in the device body 10.

The cuff 20 is a strip-shaped member used by being wound around a blood pressure measurement part, for example, an upper arm of the subject and internally includes the air bag 21. The air bag 21 communicates with the pressure sensor 22, the pump 24, and the pressure release valve 26 via the air hose 28. The air bag 21 expands in a way that air is sent into its internal space from the pump 24 and contracts in a way that the air in the internal space is released through the pressure release valve 26. The air bag 21 of the cuff 20 internally has an air pressure (a cuff pressure) detected by the pressure sensor 22.

The pressure sensor 22, which is, for example, a pressure-electricity converter using a semiconductor pressure sensor, is provided to the air hose 28. The pressure sensor 22, which converts the air pressure (the cuff pressure) of the air bag 21 of the cuff 20 into an electrical signal, has a capacitance value that varies depending on the cuff pressure. The oscillation circuit 23 outputs a signal (a pressure signal) having an oscillation frequency corresponding to the capacitance value of the pressure sensor 22 to the CPU 11. The CPU 11 generates cuff pressure data based on the signal acquired from the oscillation circuit 23. The cuff pressure data shows a waveform of the cuff pressure. For example, a pulse wave component as a signal component representing a pulse wave of the subject is superimposed on the waveform of the cuff pressure at the time of the blood pressure measurement. The CPU 100 measures a minimum blood pressure and a maximum blood pressure of the subject based on this cuff pressure data.

The pump 24 increases the cuff pressure by supplying the air bag 21 of the cuff 20 with the air through the air hose 28. The pump driving circuit 25, which controls driving of the pump 24 by outputting a drive signal to the pump 24 in accordance with a control signal from the CPU 11, starts and stops air supply from the pump 24 to the cuff 20.

The pressure release valve 26, which is, for example, an electromagnetic valve, is provided to the air hose 28. The pressure release valve 26 blocks air release from the air bag 21 of the cuff 20 while the valve is closed and releases the air in the air bag 21 of the cuff 20 through the air hose 28 while the valve is open. The valve driving circuit 27, which controls driving of the pressure release valve 26 in accordance with the control signal from the CPU 11, adjusts a degree of opening of the pressure release valve 26.

The CPU 11 may generate the control signal with respect to the pump driving circuit 25 and the valve driving circuit 27 and process the cuff pressure data acquired by the pressure sensor 22 like measuring the blood pressure by a common oscillometric method. Specifically, the CPU 11 sends the air into the cuff 20, presses a blood vessel of the subject by increasing the cuff pressure, and blocks a flow of blood. Thereafter, as the CPU 11 gradually decreases the cuff pressure, the pressure of the blood exceeds the pressure of the cuff, and from this time point, the blood starts intermittently flowing in accordance with a heartbeat (a pulse). In the oscillometric method, in a process of decreasing the pressure of the cuff after increasing it, a vibration of a blood vessel wall that is synchronous with the heartbeat (the pulse) by the timing is regarded as a variation of the cuff pressure (a pressure pulse wave). The CPU 11 measures a blood pressure value of the subject by measuring an amount of variation of the cuff pressure at the timing corresponding to the heartbeat. Generally, a cuff pressure when the pulse wave has rapidly increased is defined as “the maximum blood pressure,” and a cuff pressure when the pulse wave has rapidly decreased is defined as “the minimum blood pressure.”

In the measurement device 100 of the present invention, a pulse oximeter is configured from the probe 30 including a light-emitting element 31 and a light-receiving element 32, and the CPU 11, a light-emitting circuit 33, and a light-receiving circuit 34 included in the device body 10. The pulse oximeter non-invasively measures a blood oxygen saturation level SpO₂ by irradiating a biological tissue having a bloodstream such as a fingertip or an ear with a light from the probe to detect the light transmitted through or reflected on the biological tissue, using a principle that a light absorption property is different between HbO₂ (hemoglobin containing oxygen) and Hb (hemoglobin without the oxygen) in blood hemoglobin depending on an optical wavelength. The pulse oximeter is configured to simultaneously measure the pulse of the subject.

The probe 30 includes the light-emitting element 31 and the light-receiving element 32, and these elements 31 and 32 are provided to, for example, a fingerstall mounted on the fingertip or the like of the subject. An example of the light-emitting element 31 is a light-emitting diode. At least two kinds of light-emitting elements 31, for example, one that emits a red light and one that generates an infrared light, are provided. The two kinds of light-emitting elements 31 are alternately driven to light with a predetermined period by the light-emitting circuit 33 in the device body 10. The light-receiving element 32 is arranged at a position opposed to the light-emitting element 31 in the probe 30. An example of the light-receiving element 32 is a silicon photodiode. The light-receiving element 32 photoelectrically converts the light transmitted through the biological tissue and inputs a light signal to the light-receiving circuit 34 in the device body 10. The light-receiving circuit 34 amplifies the light signal acquired from the light-receiving element 32 to input it to the CPU 11.

The CPU 11 acquires a ratio of change rates of the red light and the infrared light based on an AC component where the red light has varied, an AC component where the infrared light has varied, a DC component where the red light does not vary, and a DC component where the infrared light does not vary. The CPU 11 reads a value of the blood oxygen saturation level (SpO₂ value) preliminarily stored in the storage unit 12 in accordance with characteristics such as a wavelength and a half-value width of the light-emitting element 31 with being associated with this ratio. Thus, the blood oxygen saturation level of the subject is measured. The CPU 11 can also measure a pulse rate per unit time of the subject based on information such as a strength variation of the light signal.

In the measurement device 100 of the present invention, a digital stethoscope is configured from the chest piece 40 including a microphone 41, and the CPU 11 and an acoustic processing circuit 42 included in the device body 10.

The chest piece 40 has a surface directly contacting the measurement part (mainly, a chest) of the subject, thus having a structure that collects the heart sound and the respiratory sound. The chest piece 40 incorporates the microphone 41. The microphone 41 converts the sound (vibration) collected at the chest piece 40 into an acoustic signal (a vibration signal) as the electrical signal to output it to the acoustic processing circuit 42 in the device body 10. The acoustic processing circuit 42, after amplifying the acoustic signal, converts it from an analog signal into a digital signal and perform a filtering process for correcting an acoustic characteristic (a frequency characteristic and a phase characteristic) on the digitized acoustic signal, thus output it to the CPU 11. The CPU 11 performs a process for determining whether the heart sound of the subject contains noise or not, for example, based on the acoustic signal acquired from the acoustic processing circuit 42.

In the measurement device 100 of the present invention, an electrocardiographic monitor is configured from the plurality of electrodes 51, 52, 53, and 54 provided to the respective cuff 20, probe 30, and chest piece 40, and the CPU 11 and an electrocardiogram processing circuit 55 included in the device body 10. The electrocardiographic monitor measures an electrocardiogram where a flow of electricity in a heart of the subject is recorded.

The plurality of electrodes include, for example, a first electrocardiograph electrode 51, an indifferent electrode 52, a second electrocardiograph electrode 53, and a third electrocardiograph electrode 54. In the example illustrated in the drawing, the first electrocardiograph electrode 51 and the indifferent electrode 52 are provided to a part contacting a skin of the subject in the cuff 20. The second electrocardiograph electrode 53 is provided to a part contacting the skin of the subject in the probe 30. Further, the third electrode 40 is provided to a part contacting the skin of the subject in the chest piece 40. Insofar as, at least, one electrocardiograph electrode 51 is provided to the cuff 20 and another one of the electrocardiograph electrodes 53 and 54 is provided to another biosignal sensor (the probe 30 or the chest piece 40), the electrocardiogram can be measured. For example, insofar as the first electrocardiograph electrode 51 and the indifferent electrode 52 are provided to the cuff 20 and the second electrocardiograph electrode 53 is provided to the probe 30, the third electrocardiograph electrode 54 of the chest piece 40 can be omitted.

The first to third electrocardiograph electrodes 51, 53, and 54 contact the measurement parts of a human body, thus functioning as electrodes for detecting physical electrical potentials of the measurement parts. Electric potential differences in the measurement parts can be derived based on electrocardiographic potentials acquired from the plurality of electrocardiograph electrodes 51, 53, and 54. The indifferent electrode 52 functions as an electrode for removing external noise induced in phase with the plurality of electrocardiograph electrodes 51, 53, and 54. The respective electrodes 51 to 54 are coupled to the electrocardiogram processing circuit 55 in the device body 10. Potential variations (the physical electrical potentials) derived from the respective electrocardiograph electrodes 51, 53, and 54 and the indifferent electrode 52 are input to the electrocardiogram processing circuit 55. The electrocardiogram processing circuit 55 differentially amplifies the physical electrical potentials derived by the respective electrocardiograph electrodes 51, 53, and 54 and removes the external noise with the derived potential from the indifferent electrode 52, thus creating an amplified electrocardiogram signal (electrocardiogram waveform). A method for creating the electrocardiogram signal may be a bipolar induction method to create the electrocardiogram using two-point electrodes as one set or a monopolar induction method to create the electrocardiogram between electrodes using the indifferent electrode as an origination, using three-point electrodes including the indifferent electrode. This amplified electrocardiogram signal is input to the CPU 11. The CPU 11 performs an analog-digital conversion on the electrocardiogram signal received from the electrocardiogram processing circuit 55, and, after performing data compression and other signal processing on the electrocardiogram signal as necessary, records the electrocardiogram signal after processing in the storage unit 12.

In the present invention, at least one electrocardiograph electrode 51 is provided to the cuff 20, and another electrocardiograph electrode making a pair with the electrocardiograph electrode 51 is provided to another biosignal sensor. For example, when the cuff 20 is wound around one arm of the subject and the probe 30 is mounted on the fingertip of another arm of the subject, the electrocardiogram signal can be created based on the electric potential difference between the first electrocardiograph electrode 51 provided to the cuff 20 and the second electrocardiograph electrode 53 provided to the probe 30. Such a configuration can improve an accuracy of the electrocardiogram signal since a distance between the first electrocardiograph electrode 51 and the second electrocardiograph electrode 53 can be sufficiently taken. What is called, a I induction can be seen based on the electric potential difference between the electrocardiograph electrodes mounted on both arms, thus being effective also in a detection of an arrhythmia.

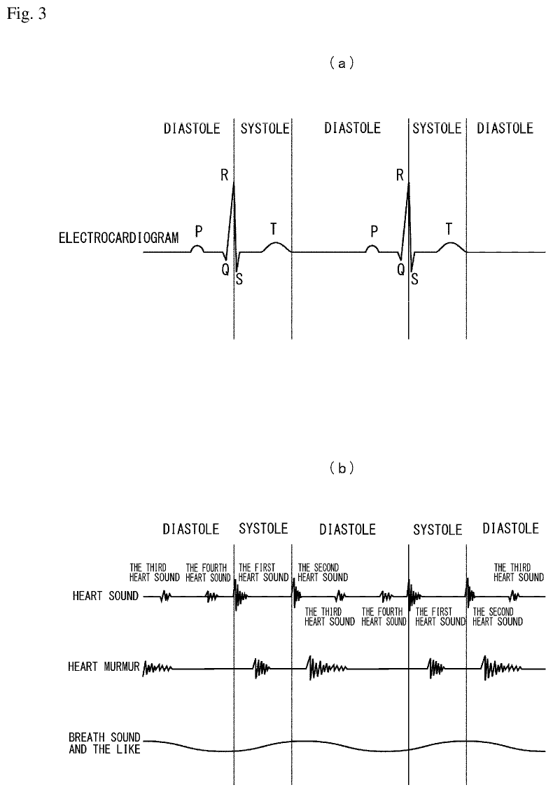

In the present invention, a heart murmur may be automatically detected based on the electrocardiogram signal measured by the electrocardiographic monitor and the acoustic signal of the heart sound measured by the digital stethoscope. FIG. 3 illustrates this mechanism. The CPU 11 accepts the electrocardiogram signal created based on physical electrical potential differences detected by the respective electrodes 51 to 54. FIG. 3A illustrates an exemplary electrocardiogram acquired based on the electrodes 51 to 54. The electrocardiogram includes a P wave, a Q wave, an R wave, an S wave, and a T wave. A period from a peak of the R wave to an end of the T wave is a systole of the heart, and a period other than it is a diastole of the heart. The CPU 11 accepts the acoustic signal sent from the microphone 41. FIG. 3B illustrates an exemplary sound around the heart detected by the microphone 41. The heart sound is a sound generated in association with the pulse of the heart, and the first heart sound, the second heart sound, the third heart sound, and the fourth heart sound are generated. Among these sounds, a sound generated immediately after the start of the systole of the heart is the first heart sound, and a sound generated at a border between the systole and the diastole is the second heart sound. The heart murmur is generated in association with the pulse of the heart but is a sound that is not generated in a normal heart. The respiratory sound and the like are normal sounds generated by an activity in the body such as respiration separately from the heart. The microphone 41 converts a sound where the heart sound, the heart murmur, the respiratory sound, and the like overlap into the electrical signal. Thus, the acoustic signal accepted by the CPU 11 contains a sound where sounds around the heart multiply overlap.

The CPU 11 extracts the systole of the heart based on the electrocardiogram signals acquired from the respective electrodes 51 to 54. Specifically, the R wave and the T wave are extracted from the electrocardiogram illustrated in FIG. 3A to define the period from the peak of the R wave to the end of the T wave as the systole. However, the second heart sound is generated at the end of the T wave. Thus, here, it is good that a time slightly before the end of the T wave is defined as an end time of the systole so as not to contain the second heart sound. Then, the CPU 11 determines whether there is the heart murmur in the extracted systole. For example, the CPU 11 detects whether there is a sound having an amplitude that exceeds a predetermined threshold between 0.3 seconds after a start of the systole and the end of the systole. The threshold can be set as necessary, for example, by acquiring it from the amplitude of the first heart sound or using an absolute value acquired in an experiment or the like. The reason why the determination is performed from 0.3 seconds after the start of the systole is to eliminate a period until the first heart sound as a large sound always existing at the start of the systole sufficiently decreases. This period is influenced by the pulse rate and the like. Thus, it is not limited to 0.3 seconds, can be appropriately changed, and may be set to vary corresponding to the pulse rate. Further, when there is a sound exceeding the threshold, the CPU 11 records its generation timing within the systole. Then, the CPU 11 determines that there is the heart murmur when, after the detection in consecutive multiple times (for example, ten times) of systoles is performed, there are the sounds exceeding the threshold at an identical timing in all the times. The reason why the determination is performed based on the multiple times of systoles is to eliminate the influence of the noise such as the respiratory sound. From the timing of respiration, for example, around ten times of measurements can eliminate the influence of the respiratory sound. This number of times is not limited to ten times and may be appropriately changed. A criterion for determination that there are the sounds exceeding the threshold in all of the ten times of systoles is also an example. A condition for determination can be also appropriately changed such as determining that there is the heart murmur even when the sounds exceed the threshold less than ten times.

In the present invention, using the electrocardiogram signal measured by the electrocardiographic monitor, an accuracy of the blood pressure measurement by the sphygmomanometer can be enhanced. The CPU 11 accepts the electrocardiogram signal created based on the physical electrical potential differences detected by the respective electrodes 51 to 54. Then, the pulse wave of the subject is extracted from this electrocardiogram signal. Specifically, the systole of the heart (the period from the peak of the R wave to the end of the T wave is the systole of the heart: see FIG. 3A) is extracted. The CPU 11, after increasing the cuff pressure in the cuff by controlling the pump 24, decreases the pressure of the pressure release valve 26, and measures the maximum blood pressure and the minimum blood pressure of the subject in a process from the pressurization to the depressurization. Here, when a sign of the arrhythmia is seen in the subject, the blood pressure may become highest at a timing other than the systole of the heart. Alternatively, when a sign of a low blood pressure is seen in the subject, the blood pressure may become lowest at a timing other than the systole of the heart. These maximum blood pressure and minimum blood pressure at the timing other than the systole of the heart cannot be said to accurately indicate the blood pressure value of the subject. Thus, the CPU 11 ignores (cancels) the maximum blood pressure and the minimum blood pressure at the timing other than the systole of the heart and measures the maximum blood pressure and the minimum blood pressure detected within the period of the systole of the heart. Thus, it is possible to enhance an accuracy of the automated sphygmomanometer by accurately acquiring the timing of the pulse wave of the subject from the electrocardiogram and acquiring the maximum blood pressure and the minimum blood pressure at the timing corresponding to pulsation.

When the probe 30 and the chest piece 40 are employed as the biosignal sensor for measuring the vital signs, these probe 30 and chest piece 40 preferably have a mechanism removably combined. The probe 30 and the chest piece 40 may be ones combinable with a physical structure such as fitting to one another or may be ones combinable with a magnetic force by mounting permanent magnets on both. This facilitates holding of the probe 30 and the chest piece 40 in one hand, for example, as illustrated in FIG. 1. In accordance with a usage situation, the probe 30 and the chest piece 40 can be separately used.

In the embodiment illustrated in FIG. 1 and FIG. 2, the auscultation chest piece 40 is employed, but instead of it or together with it, a thermometer (not illustrated) for measuring a body temperature of the subject may be employed. In this case, one or more electrocardiogram measurement electrodes are provided to a part contacting the skin of the subject in the thermometer.

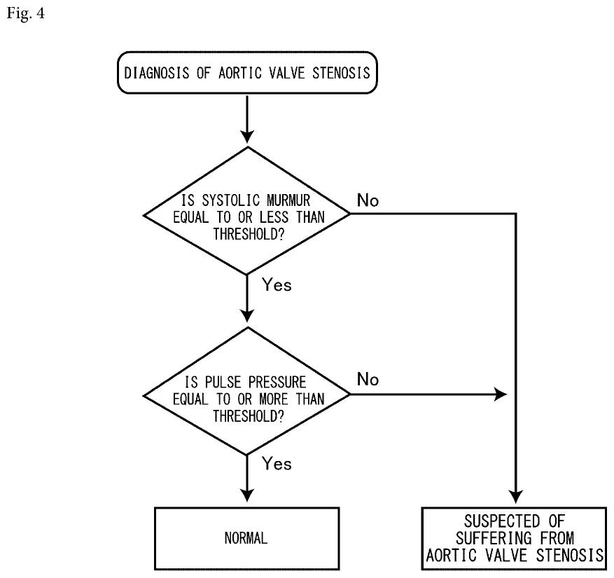

FIG. 4 illustrates an exemplary automatic diagnosis flow for an aortic valve stenosis. The CPU of the device body 10 discriminates time slots of the systole and the diastole of the heart of the subject from the electrocardiogram and acquires a difference (that is, “the pulse pressure”) in the blood pressures of the subject in the systole and the diastole. As described above, when the heart murmur is recognized in the systole, the subject is strongly suspected of suffering from the aortic valve stenosis. This disease has a trend that the heart murmur in the systole increase as the symptom gets worse, but the heart murmur in the systole rather becomes weak as the symptom further becomes severe. On the other hand, a patient suffering from an end-stage aortic valve stenosis has a trend that the pulse pressure decreases. Accordingly, simultaneously measuring the pulse pressure in addition to the heart murmur in the systole ensures a more certain diagnosis even in a case of the severe aortic valve stenosis.

That is, as illustrated in FIG. 4, when the systolic murmur is equal to or less than a constant threshold and the pulse pressure is equal to or more than a constant threshold, it is diagnosed that the subject is normal. On the other hand, when the systolic murmur exceeds the constant threshold, it is diagnosed that the subject is suspected of suffering from the aortic valve stenosis. Even when the systolic murmur is equal to or less than the constant threshold, in a case where the pulse pressure is less than the constant threshold, it is diagnosed that the subject is suspected of suffering from the aortic valve stenosis. The patient of the aortic valve stenosis has a trend that the pulse pressure decreases, and in an extreme case, the blood pressure is 120 mmHg (the systole)/110 mmHg (the diastole), thus having a significant small difference (that is, the pulse pressure) between a systolic blood pressure and a diastolic blood pressure. In this case, even when the systolic murmur is equal to or less than the threshold, it can be diagnosed that the subject is suspected of suffering the aortic valve stenosis. The threshold of the systolic murmur and the threshold of the pulse pressure may be appropriately adjusted.

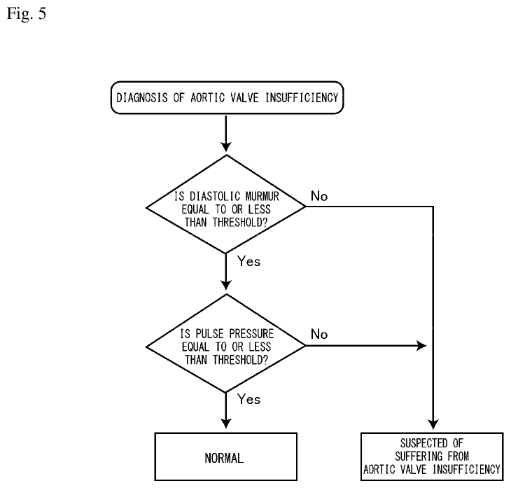

FIG. 5 illustrates an exemplary automatic diagnosis flow for an aortic valve insufficiency. The CPU of the device body 10 discriminates time slots of the systole and the diastole of the subject from the electrocardiogram and acquires a difference (that is, “the pulse pressure”) in the blood pressures of the subject in the systole and the diastole. When the heart murmur is recognized in the diastole, the subject is strongly suspected of suffering from the aortic valve insufficiency. This disease has a trend that the heart murmur in the diastole increases as the symptom gets worse, but the heart murmur in the diastole rather becomes weak as the symptom further becomes severe. On the other hand, a patient suffering from an end-stage aortic valve insufficiency has a trend that the pulse pressure increases. Accordingly, simultaneously measuring the pulse pressure in addition to the heart murmur in the diastole ensures a more certain diagnosis even in a case of the severe aortic valve insufficiency.

That is, as illustrated in FIG. 5, when the diastolic murmur is equal to or less than a constant threshold and the pulse pressure is equal to or less than a constant threshold, it is diagnosed that the subject is normal. On the other hand, when the diastolic murmur exceeds the constant threshold, it is diagnosed that the subject is suspected of suffering from the aortic valve insufficiency. Even when the diastolic murmur is equal to or less than the constant threshold, in a case where the pulse pressure exceeds the constant threshold, it is diagnosed that the subject is suspected of suffering from the aortic valve insufficiency. The threshold of the systolic murmur and the threshold of the pulse pressure may be appropriately adjusted.

In the present description, the embodiment of the present invention has been described above by referring to the drawings to express the content of the present invention. However, the present invention is not limited to the above-described embodiment and encompasses changed forms and improved forms obvious for those skilled in the art based on the matters described in the present description.

### DESCRIPTION OF REFERENCE SIGNS

10 . . . device body
11 . . . CPU
12 . . . storage unit
13 . . . display unit
14 . . . operation unit
20 . . . cuff
21 . . . air bag
22 . . . pressure sensor
23 . . . oscillation circuit
24 . . . pump
25 . . . pump driving circuit
26 . . . pressure release valve
27 . . . valve driving circuit
28 . . . air hose
30 . . . probe
31 . . . light-emitting element
32 . . . light-receiving element
33 . . . light-emitting circuit
34 . . . light-receiving circuit
40 . . . chest piece
41 . . . microphone
42 . . . acoustic processing circuit
51 . . . first electrocardiograph electrode
52 . . . indifferent electrode
53 . . . second electrocardiograph electrode
54 . . . third electrocardiograph electrode
55 . . . electrocardiogram processing circuit
100 . . . vital sign measurement device

1. A vital sign measurement device comprising:
a cuff for measuring blood pressure that presses on a certain measurement part of a subject;
one or a plurality of biosignal sensors that detect a biosignal at another measurement part of the subject;
a plurality of electrodes that contact a skin of the subject and detect physical electrical potentials; and
a device body,
wherein the device body:
measures a blood pressure of the subject by increasing and decreasing a cuff pressure in the cuff;
measures a vital sign other than the blood pressure or an electrocardiogram of the subject based on the biosignal detected by the biosignal sensor; and
measures the electrocardiogram of the subject based on the physical electrical potentials detected by the plurality of electrodes,
wherein at least one of the plurality of electrodes is provided to the cuff, and
wherein at least one of the plurality of electrodes is provided to the biosignal sensor.

2. The vital sign measurement device according to claim 1, wherein
the biosignal sensor includes a probe that irradiates a biological tissue having a bloodstream of the subject with a light to detect optical information of a transmitted light or a reflected light, and
the device body measures at least any one of a blood oxygen saturation level and a pulse of the subject based on the optical information detected by the probe.

3. The vital sign measurement device according to claim 2, wherein
the biosignal sensor further includes a thermometer.

4. The vital sign measurement device according to claim 2, wherein
the biosignal sensor includes a chest piece including a microphone that converts a heart sound of the subject into an electrical signal.

5. The vital sign measurement device according to claim 4, wherein
any one of the plurality of electrodes is provided to a part contacting the skin of the subject in the probe, and
any one of the plurality of electrodes is provided to a part contacting the skin of the subject in the chest piece.

6. The vital sign measurement device according to claim 4 or 5, wherein
the probe and the chest piece are removably combined.

7. The vital sign measurement device according to claim 4, wherein
the device body extracts one time slot of both or any one of a systole and a diastole of a heart of the subject from the electrocardiogram and determines whether there is a heart murmur in the heart sound in the time slot extracted from a heart sound signal acquired by the microphone.

8. The vital sign measurement device according to claim 7, wherein
the device body discriminates the time slots of the systole and the diastole of the heart of the subject from the electrocardiogram and acquires a difference in the blood pressures of the subject in the systole and the diastole.
